(19) United States
(12) Patent Application Publication
(10) Pub. No.: US 2005/0054652 A1
(43) Pub. Date: Mar. 10, 2005

(54) Methods of treating metabolic syndrome using dopamine receptor agonists

(76) Inventor: Anthony H. Cincotta, Tiverton, RI (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/944,631

(22) Filed: Sep. 17, 2004

Related U.S. Application Data
(63) Continuation-in-part of application No. 10/821,233, filed on Apr. 8, 2004.
Application No. 10/821,233 is a continuation-in-part of application No. 10/627,014, filed on Jul. 25, 2003.
(60) Provisional application No. 60/399,180, filed on Jul. 29, 2002.

(51) Int. Cl. A61K 31/498; A61K 31/517; A61K 31/4745; A61K 31/137

(52) U.S. Cl. 514/250; 514/266.1; 514/295; 514/649

(57) ABSTRACT

The present invention is directed to a method of simultaneously treating hypertension, hypertriglyceridemia, a pro-inflammatory state, a pro-coagulative state, and insulin resistance (with or without treating obesity or endothelial dysfunction), associated with or independent from Metabolic Syndrome, comprising the step of administering to a patient suffering from such disorders a therapeutically effective amount of a central acting dopamine agonist. In one embodiment, the central acting dopamine agonist is bromocriptine, optionally combined with a pharmaceutically acceptable carrier.

### CROSS-REFERENCE TO RELATED APPLICATIONS

This application is a Continuation-in-Part of U.S. application Ser. No. 10/821,233 filed Apr. 8, 2004, which is a Continuation-in-Part of U.S. application Ser. No. 10/627,014, filed Jul. 25, 2003, which claims the benefit of U.S. Provisional Application Ser. No. 60/399,180 filed Jul. 29, 2002.

### BACKGROUND OF THE INVENTION

1. Field of the Invention

This invention relates to methods of treating metabolic disorders, and more particularly, to method of treating Metabolic Syndrome, or its composite individual disorders by administering a central acting dopamine agonist such as bromocriptine.

2. Description of the Related Art

Metabolism is a complex orchestration of biochemical processes among cells and tissues of the body all working in concert to ensure the survival of the organism as a whole. The central nervous system plays a major role in integrating these metabolic activities to maintain normal biological homeostasis within the body. Environmental and genetic perturbations to this central nervous system control of metabolism can manifest as a range of metabolic disorders. Additionally, since metabolic processes have profound effects on the entire body, diseases and disorders affecting metabolism generally affect other areas of the body as well. For example, individuals suffering from Type 2 diabetes often experience problems with other body organs and systems. Typically, plasma glucose levels are elevated in Type 2 diabetes as a result of the body's resistance to the glucose-lowering effects of a hormone called insulin. Type 2 diabetes is associated with damage to various organs such as the eyes, nerves, and kidneys. The disease is also associated with substantially increased risk for cardiovascular disease, the leading cause of death in Type 2 diabetics. The prevalence of Type 2 diabetes is reaching epidemic proportions in the United States and around the world.

According to the guidelines of the American Diabetes Association, to be diagnosed with Type 2 diabetes, an individual must have a fasting plasma glucose level greater than or equal to 126 mg/dl or a 2-hour oral glucose tolerance test (OGTT) plasma glucose value of greater than or equal to 200 mg/dl (Diabetes Care, 26:S5-S20, 2003). A related condition called pre-diabetes is defined as having a fasting glucose level of greater than 100 mg/dl but less than 126 mg/dl or a 2-hour OGTT plasma glucose level of greater than 140 mg/dl but less than 200 mg/dl. Mounting evidence suggests that the pre-diabetes condition may be a risk factor for developing cardiovascular disease (Diabetes Care 26:2910-2914, 2003).

Metabolic Syndrome, also referred to as Syndrome X, is another metabolic disorder that affects other pathways and systems in the body. Originally, Metabolic Syndrome was defined as a cluster of metabolic disorders (including obesity, insulin resistance, hypertension, and dyslipidemia primarily hypertriglyceridemia), that synergize to potentiate cardiovascular disease. More recently, the U.S. National Cholesterol Education Program has classified Metabolic Syndrome as meeting three out of the following five criteria: fasting glucose level of at least 110 mg/dl, plasma triglyceride level of at least 150 mg/dl (hypertriglycerdemia), HDL cholesterol below 40 mg/dl in men or below 50 mg/dl in women, blood pressure at least 130/85 mm Hg (hypertension), and central obesity, with central obesity being defined as abdominal waist circumference greater than 40 inches for men and greater than 35 inches for women. The American Diabetes Association estimates that 1 in every 5 overweight people suffer from Metabolic Syndrome.

While these disorders and diseases are related, it is clear that they have individual and distinct pathologies. For that reason, drugs used to treat one disorder may not be effective against another disorder. For instance, drugs that are effective in treating Type 2 diabetes or pre-diabetes have little to no effect on Metabolic Syndrome. Additionally, certain drugs used to treat Type 2 diabetes or pre-diabetes may increase blood pressure (hypertension) or cause weight gain in the individuals taking the medication. For example, thiazolidinediones used in the treatment of Type 2 diabetes cause weight gain and has marginal effects on hypertension. Another anti-diabetic agent, metformin, also has marginal effects on hypertension and hypertriglyceridemia. Insulin, which is a hormone used to treat Type 2 diabetes can potentiate hypertension and weight gain. Moreover, anti-hypertensive drugs do not necessarily treat dyslipidemia or obesity, and many can worsen insulin sensitivity instead of improving it.

Since the Metabolic Syndrome is diagnosed as having several criteria (as described above) yet also encompasses vascular abnormalities such as endothelial dysfunction, vascular pro-inflammatory condition, and vascular pro-coagulative condition, the treatment of Metabolic Syndrome according to the present invention further includes
a. Treatment of endothelial dysfunction associated with cardiovascular disease;
b. Treatment of hypertension, vascular pro-inflammatory state, and pro-coagulative state simultaneously. Examples of pro-inflammatory state blood markers include but are not limited to: C-reactive protein, serum amyloid A protein, interleukin-6, interleukin-1, Tumor Necrosis Factor-alpha, homocysteine, and white blood cell count. Examples of pro-coagulative state blood markers include but are not limited to: hematocrit viscosity, red cell aggregation, plasminogen activator inhibitor-1, fibrinogen, van Willebrand factor, Factor VII, Factor VIII, and Factor IX;
c. Treatment of at least two of hypertension, vascular pro-inflammatory state, or pro-coagulative state simultaneously; and
d. Treatment of at least one of hypertension, vascular pro-inflammatory state, or pro-coagulative state.

The endothelium can modify circulating factors as well as synthesize and release factors that influence cardiovascular health and disease. Endothelium dysfunction is characterized by alterations in endothelium modulation of the vasculature that favor or potentiate vasoconstriction, a pro-coagulant state, and/or a pro-inflammatory state as well as other biochemical process that all contribute to the initiation and progression of atherosclerosis (Am. J. Cardiol. 91(12A): 3H-1H, 2003; Am. J, Cardiol. 115 Suppl 8A:99S-106S, 2003)

A variety of treatments are available for diseases associated with obesity, including Type 2 Diabetes. For example, U.S. Pat. No. 6,506,799 discloses methods of treating cardiovascular diseases, dyslipidemia, dyslipoproteinemia, and hypertension comprising administering a composition comprising an ether compound.

U.S. Pat. No. 6,441,036 discloses fatty acid analogous which can be used for the treatment and/or prevention of obesity, fatty liver and hypertension.

U.S. Pat. No. 6,410,339 discloses use of cortisol agonist for preparing a system for diagnosis of the Metabolic Syndrome and related conditions as belly fatness, insulin resistance including increased risk of developing senile diabetes, i.e., diabetes type II, high blood fats and high blood pressure, in which system the dose of cortisol agonist is in an interval where a difference is obtained in the inhibitory effect of the autoproduction of cortisol in individuals suffering from the Metabolic Syndrome, compared to normal values.

U.S. Pat. No. 6,376,464 discloses peptides and peptide analogues that mimic the structural and pharmacological properties of human ApoA-I. The peptides and peptide analogues are useful to treat a variety of disorders associated with dyslipidemia.

U.S. Pat. No. 6,322,976 discloses, among other things, methods of diagnosing a disease associated with a defect in insulin action, glucose metabolism, fatty acid metabolism, and/or catecholamine action by detecting a mutation in the CD36 gene.

U.S. Pat. No. 6,197,765 discloses a treatment for Metabolic Syndrome (syndrome-X), and resulting complications, by administration of diazoxide.

U.S. Pat. No. 6,166,017 discloses a method for the medical treatment of diabetes mellitus type II and for counteracting the risk factors forming part of the Metabolic Syndrome by administration of ketoconazole.

U.S. Pat. No. 6,040,292 discloses methods for the treatment of diabetes mellitus, including type I, type II, and insulin resistant diabetes (both type I and type II). The methods of the invention employ administration of rhIGF-I/IGFBP-3 complex to a subject suffering from the symptoms of diabetes mellitus. Administration of rhIGF-I/IGFBP-3 to a subject suffering from the symptoms of diabetes mellitus results in amelioration or stabilization of the symptoms of diabetes.

U.S. Pat. No. 5,877,183 discloses methods for the regulation and modification of lipid and glucose metabolism, but not Metabolic Syndrome, by administering to a subject a dopamine D1 agonist, optionally in combination with a dopamine D2 agonist, an alpha-1 adrenergic antagonist, an alpha-2 adrenergic agonist, or a serotonergic inhibitor, or optionally in combination with a dopamine D2 agonist coadministered with at least one of alpha-1 adrenergic antagonist, an alpha-2 adrenergic agonist, or a serotonergic inhibitor, and further administering the subject a serotonin 5HT_{1b} agonist. It is well known that dopamine agonists function to both activate and deactivate dopamine receptors and thereby reduce dopaminergic neuronal activity.

U.S. Pat. No. 5,741,503 discloses methods for regulating or ameliorating lipid metabolism which comprise administration or timed administration of inhibitors of dopamine beta hydroxylase (DBH). However, the focus of this technology is reduction in noradrenergic neuronal activity level only and does not increase dopaminergic neuronal activity inasmuch as DBH is not present in dopaminergic neurons that are anatomically distinct from noradrenergic neurons where DBH resides.

In addition, several U.S. Patents disclose use of dopamine agonists such as bromocriptine for use in treating pathologies relating to Type II diabetes. See, for example, U.S. Pat. Nos. 6,004,972; 5,866,584; 5,756,513; and 5,468,755.

A significant complicating issue in the treatment of metabolic disorders is that the individual pathologies of Metabolic Syndrome differ in their nature and magnitude whether presented alone or as part of the syndrome because the pathologies of the syndrome tend to synergize to produce increased risk of morbidity and mortality (Reviewed in GM Reaven, Diabetes, Obesity, and Metabolism, 4: (Suppl. 1) S13-S-18, 2002). In other words, a Metabolic Syndrome subject carries a different increased risk of cardiovascular disease as a result of his/her hypertension than does a hypertensive subject without Metabolic Syndrome. Currently, the U.S. Food and Drug Administration has not approved the use of any drug for the treatment of Metabolic Syndrome. However, inasmuch as this syndrome affects at least 20% of overweight people and is a serious risk factor for cardiovascular disease, an effective treatment for the disorder is needed. The present invention is believed to be an answer to that need.

### BRIEF SUMMARY OF THE INVENTION

In one aspect, the present invention is directed to a method of simultaneously treating hypertension, hypertriglyceridemia, a pro-inflammatory state, and insulin resistance associated with Metabolic Syndrome, the method comprising the step of administering to a patient suffering with Metabolic Syndrome a therapeutically effective amount of a central acting dopamine agonist to simultaneously treat hypertension, hypertriglyceridemia, a pro-inflammatory state, and insulin resistance.

In another aspect, the present invention is directed to a method of simultaneously treating hypertension, hypertriglyceridemia, a pro-inflammatory state, and insulin resistance associated with Metabolic Syndrome, the method comprising the step of administering to a patient suffering with Metabolic Syndrome a therapeutically effective amount of a pharmaceutical composition comprising bromcriptine and a pharmaceutically acceptable carrier to simultaneously treat hypertension, hypertriglyceridemia, a pro-inflammatory state, and insulin resistance.

In another aspect, the present invention is directed to a method for simultaneously treating hypertension, hypertriglyceridemia, a pro-inflammatory state, a pro-coagulative state, and insulin resistance associated with the Metabolic Syndrome, the method comprising the step of administering to a patient suffering from Metabolic Syndrome a therapeutically effective amount of a central acting dopamine agonist to simultaneously treat hypertension, hypertriglyceridemia, a pro-inflammatory state, a pro-coagulative state, and insulin resistance.

In another aspect, the present invention is directed to a method for simultaneously treating hypertension, a pro-inflammatory state, a pro-coagulative state, and a pro-oxidant state associated with the Metabolic Syndrome, the method comprising the step of: administering to a patient suffering from Metabolic Syndrome a therapeutically effective amount of a central acting dopamine agonist to simultaneously treat hypertension, a pro-inflammatory state, a pro-coagulative state, a pro-oxidant state, and any combination thereof.

In another aspect, the present invention is directed to a method for simultaneously treating hypertension, a pro-inflammatory state, and a pro-coagulative state the method comprising the step of: administering to a patient suffering from hypertension, a pro-inflammatory state, and a pro-coagulative state, a therapeutically effective amount of a central acting dopamine agonist to simultaneously treat hypertension, a pro-inflammatory state, a pro-coagulative state, a pro-oxidant state, and any combination thereof.

In another aspect, the present invention is directed to a method for treating at least one of hypertension, a pro-inflammatory state, and a pro-coagulative state, or a pro-oxidant state associated with the Metabolic Syndrome, the method comprising the step of administering to a patient suffering from Metabolic Syndrome a therapeutically effective amount of a central acting dopamine agonist to treat at least one of hypertension, a pro-inflammatory state, a pro-coagulative state, and a pro-oxidant state.

In another aspect, the present invention is directed to a method for treating at least one of hypertension, a pro-inflammatory state, and a pro-coagulative state the method comprising the step of administering to a patient suffering from at least one of hypertension, a pro-inflammatory state, and a pro-coagulative state, a therapeutically effective amount of a central acting dopamine agonist to treat at least one of hypertension, a pro-inflammatory state, and a pro-coagulative state.

In another aspect, the present invention is directed to a method for treating endothelial dysfunction associated with the Metabolic Syndrome, the method comprising the step of administering to a patient suffering from Metabolic Syndrome a therapeutically effective amount of a central acting dopamine agonist to treat endothelial dysfunction.

In another aspect, the present invention is directed to a method for treating endothelial dysfunction associated with cardiovascular disease, the method comprising the step of administering to a patient suffering from endothelial dysfunction, a therapeutically effective amount of a central acting dopamine agonist to treat endothelial dysfunction.

These and other aspects will be described in more details in the following detailed description of the invention.

### DETAILED DESCRIPTION

In accordance with the present invention, a novel treatment for the Metabolic Syndrome (obesity, insulin resistance, hyperlipidemia, and hypertension) and Type 2 diabetes is presented. The treatment method of the invention also encompasses treating one or more of hypertension, hypertriglyceridemia, a pro-inflammatory state, a pro-coagulative state, insulin resistance, and/or a pro-oxidant state independently or associated with the Metabolic Syndrome. The treatment method of the invention also encompasses treating endothelial dysfunction associated with the Metabolic Syndrome or cardiovascular disease. The treatment methods comprise administering to a mammalian species in need of such treatment a pharmaceutical composition that simultaneously stimulates an increase in central dopaminergic neuronal activity level (particularly within neurons innervating the hypothalamus and the hypothalamus itself) and a decrease in central noradrenergic neuronal activity level (particularly within the brain stem region that innervates the hypothalamus and the hypothalamus itself). It has been unexpectedly discovered that increasing the ratio of dopaminergic neuronal to noradrenergic neuronal activity within the hypothalamus of the central nervous system improves the Metabolic Syndrome and/or Type 2 diabetes conditions, as well as the conditions of hypertension, hypertriglyceridemia, pro-inflammatory states, pro-coagulative states, pro-oxidant states, insulin resistance, and endothelial dysfunction associated with or independent from the Metabolic Syndrome. As defined herein, “neuronal activity” refers to either an increase or decrease in the synaptic neurochemical signal transmission of a neuron to another thereby affecting action potential. As defined herein, the term “pro-oxidant state” refers toan increase in the oxidizing capacity of components or molecular species within the blood or tissues.

An important advantage of the present invention is avoidance of desensitization. Prior treatments result in the neuronal activity becoming “sensitized” to the application of drugs, and ultimately lead to ineffectiveness of these treatments. By contrast, the present invention minimizes desensitization of stimulation of dopaminergic neurons or of inhibition of noradrenergic neurons, and thus makes the treatments highly effective.

In one embodiment, the method of the present invention includes administering to a subject in need of treatment for the Metabolic Syndrome or Type 2 diabetes a pharmaceutical composition comprising (1) at least one compound that stimulates an increase in central dopaminergic neuronal activity level in said subject, and (2) at least one compound that stimulates a decrease in central noradrenergic neuronal activity level in said subject. In an alternative embodiment, the pharmaceutical composition may include a single compound that stimulates an increase in central dopaminergic neuronal activity level as well as stimulates a decrease in central noradrenergic neuronal activity level. It is also contemplated that two, three, four, or more such compounds, each capable of simultaneously stimulating an increase in central dopaminergic neuronal activity level as well as stimulates a decrease in central noradrenergic neuronal activity level, may be used in the pharmaceutical composition. In all embodiments, however, the ratio of dopaminergic neuronal to noradrenergic neuronal activity within the hypothalamus is increased.

The increase in central dopaminergic neuronal activity level can take place by any mechanism. In preferred embodiments, the increase in central dopaminergic neuronal activity level occurs by including in the pharmaceutical composition at least one compound that stimulates an increase in central dopaminergic neuronal activity level. Preferably, such compounds include, but are not limited to, dopamine reuptake inhibitors, dopamine presynaptic transporter inhibitors, presynaptic dopamine release enhancers, post synaptic dopamine receptor agonists, dopamine synthesis stimulators, and/or dopamine catabolism inhibitors. Examples of useful compounds that stimulate an increase in central dopaminergic neuronal activity level include, but are not limited to, GBR-12935 (known as 1-[2-(diphenylmethoxy)ethyl]-4-(3-phenylpropyl)piperazine); BDNF (Brain Derived Neurotrophic Factor), quinpirole ((4aR-trans)-4,4a,5,6,7,8,8a,9-octahydro-5-propyl-1H-pyrazolo[3,4-g]quinoline); SKF38393 (1-phenyl-7,8-dihydroxy-2,3,4,5-tetrahydro-1H-3-benzazepine hydrochloride); deprenyl (also known as “Selegiline”); apomorphine, pramipexole (sold commercially under the name “Mirapex”), GBR-12909 (“Vanoxerine”, 1-2-(bis(4-fluorophenyl)-methoxy)-ethyl-4-(3-phenylpropyl)piperazine); and combinations thereof.

The inhibition of noradrenergic neuronal activities may also be accomplished via any mechanism. In preferred embodiments, stimulation of a decrease in central noradrenergic activity level occurs by administration of at least one compound that results in a decrease in central noradrenergic activity level. Preferably, such compounds include, but are not limited to, postsynaptic noradrenergic receptor blockade compounds, inhibitors of noradrenalin release, inhibitors of noradrenalin synthesis, activators of noradrenalin presynaptic reuptake, and activators of noradrenalin catabolism presynaptically and in the synapse. Examples of useful compounds that decrease central noradrenergic activity level include, but are not limited to, prazosin (1-(4-amino-6,7-dimethoxy-2-quinazolinyl)-4-(2-furanylcarbonyl)piperizine): propranolol (1-(isopropylamino)-3-(1-naphthyloxy)-2-propanol); clonidine (2-(2,6-dichloroanilino)-2-imidazoline); fusaric acid (5-butyl-2-pyridinecarboxylic acid; 5-butylpicolinic acid); dopamine; phenoxybenzamine; phentolamine, (3-[[(4,5-dihydro-1H-imidazol-2-yl)methyl](4-methylphenyl)amino]phenol; 2-[N-(m-hydroxyphenyl-p-toluidineomethyl)imidazoline); guanfacine (sold under the brand name “Tenex”); and combinations thereof.

As indicated above, the method of the invention may also include administration of a pharmaceutical composition that includes a single or individual compound that simultaneously stimulates an increase in central dopaminergic neuronal activity level and a decrease in central noradrenergic neuronal activity level. Examples of such compounds include catecholamine modifiers, such as dopamine.

The compounds of the invention are preferably administered internally, e.g., orally, subcutaneously, transdermally, sublingually or intravenously, in the form of conventional pharmaceutical compositions, for example in conventional enteral or parenteral pharmaceutically acceptable excipients containing organic and/or inorganic inert carriers, such as water, gelatin, lactose, starch, magnesium stearate, talc, plant oils, gums, alcohol, Vaseline, or the like. The pharmaceutical compositions can be in conventional solid forms, for example, tablets, dragees, suppositories, capsules, or the like, or conventional liquid forms, such as suspensions, emulsions, or the like. If desired, they can be sterilized and/or contain conventional pharmaceutical adjuvants, such as preservatives, stabilizing agents, wetting agents, emulsifying agents, buffers, or salts used for the adjustment of osmotic pressure. The pharmaceutical compositions may also contain other therapeutically active materials. The pharmaceutical compositions of the invention can be made using conventional methods know in the art of pharmaceutical manufacturing.

The pharmaceutical compositions of the invention should include an amount of the compound(s) of the invention effective for treatment of the Metabolic Syndrome or Type 2 diabetes. The effective dosage will depend on the severity of the diseases and the activity of the particular compound(s) employed, and is thus within the ordinary skill of the art to determine for any particular host mammal or other host organism. Suitable dosages may be, for example, in the range of about 0.001 to about 100 mg per kg for a human being, and more preferably from about 0.1 to about 50 mg per kg for a human being.

The ratio of the compound(s) that stimulates an increase in central dopaminergic neuronal activity level to the compound(s) that stimulates a decrease in central noradrenergic neuronal activity level in the pharmaceutical composition generally ranges from about 500:1 to 1:500 on a weight-to-weight basis (w:w), and more preferably from about 100:1 to 1:100 on a weight-to-weight basis (w:w).

In further accordance with the method of the present invention, it has been surprisingly found that one or more of the metabolic disorders associated with Metabolic Syndrome may be treated by administering a central acting dopamine agonist, in particular hypertension, hypertriglyceridemia, a pro-inflammatory state, insulin resistance, and, optionally, obesity. Dopamine agonists have been used to treat diseases such as Parkinson's disease and diabetes. However, it has been surprisingly found that administering dopamine agonists to patients suffering from Metabolic Syndrome will alleviate their symptoms. An important advantage of the present invention is the ability to simultaneously treat multiple disorders of the Syndrome such as hypertension, insulin resistance, hypertriglyceridemia, a pro-inflammatory state, and optionally obesity.

As indicated above, in one embodiment, the present invention is directed to a method of treating insulin resistance, hypertension, a pro-inflammatory state, and hypertriglyceridemia. Fasting glucose of at least 110 mg/dl, plasma triglycerides at least 150 mg/dl, HDL cholesterol below 40 mg/dl in men or below 50 mg/dl in women, blood pressure at least 130/85 mm Hg, are also symptoms indicative of Metabolic Syndrome.

According to the method of the invention, treatment of one or more of the metabolic disorders associated with Metabolic Syndrome includes administering to a patient suffering from Metabolic Syndrome a therapeutically effective amount of a central acting dopamine agonist. Preferred central acting dopamine agonists include bromocriptine, quinpirole, quinerolane, talipexole, ropinirole, apomorphine, lisuride, terguride, fenoldopam, and combinations thereof. A most preferred central acting dopamine agonist is bromocriptine.

In accordance with the method of the invention, the central acting dopamine agonist is preferably administered internally, e.g., orally sublingually, or intravenously, in the form of conventional pharmaceutical compositions, for example in conventional enteral or parenteral pharmaceutically acceptable excipients containing organic and/or inorganic inert carriers, such as water, gelatin, lactose, starch, magnesium stearate, talc, plant oils, gums, alcohol, petroleum jelley, or the like. The pharmaceutical compositions can be in conventional solid forms, for example, tablets, dragees, suppositories, capsules, or the like, or conventional liquid forms, such as suspensions, emulsions, or the like. If desired, they can be sterilized and/or contain conventional pharmaceutical adjuvants, such as preservatives, stabilizing agents, wetting agents, emulsifying agents, buffers, or salts used for the adjustment of osmotic pressure. The pharmaceutical compositions may also contain other therapeutically active materials. The pharmaceutical compositions of the invention can be made using conventional methods know in the art of pharmaceutical manufacturing.

Further in accordance with the method of the present invention, the compounds or pharmaceutical compositions should include an amount of central acting dopamine agonist that is effective for treatment of the Metabolic Syndrome, or hypertension, hypertriglyceridemia, a pro-inflammatory state, a pro-coagulative state, insulin resistance, or endothelial dysfunction, either associated with the Metabolic Syndrome or independent of it. The effective dosage of pharmaceutical composition and/or central acting dopamine agonist will depend on the severity of the diseases and the activity of the particular compound(s) employed, and is thus within the ordinary skill of the art to determine for any particular host mammal or other host organism. Suitable dosages of central acting dopamine agonist may be, for example, in the range of about 0.001 to about 0.2 mg per kg for a human being, and more preferably from about 0.01 to about 0.05 mg per kg for a human being. For oral tablets, the ratio of bromocriptine to carriers on a weight by weight basis is about 1 mg bromocriptine per 90 mg of tablet.

### EXAMPLE 1

Four different groups of animals exhibiting the Metabolic Syndrome and/or Type 2 diabetes are treated with either saline as control, central dopamine neuronal activity activator(s), central noradrenergic neuronal activity inhibitor(s), or a molecular entity or entities that is/are both a central dopaminergic neuronal activity activator and central noradrenergic neuronal activity inhibitor, respectively.

Relative to the control group the dopaminergic neuronal activator/noradrenergic neuronal activity inhibitor group exhibits the greatest improvement in metabolism (decrease in obesity, dyslipidemia, hypertension, insulin resistance, hyperinsulinemia, and/or hyperglycemia) that is also significantly better than that of either the dopaminergic activator or noradrenergic inhibitor groups. An unexpected synergism between the dopaminergic neuronal activity stimulator(s) and noradrenergic neuronal activity inhibitors(s) is observed relative to the effects on improvement of the Metabolic Syndrome and/or Type 2 diabetes.

### EXAMPLE 2

Two groups of animals exhibiting the Metabolic Syndrome are treated with either a dopamine agonist such as bromocriptine or vehicle (control) for a period of time of approximately two weeks. The insulin sensitivity, plasma triglyceride level, blood pressure, pro-coagluant and pro-inflammatory factor level(s) of the animals are then determined. Relative to the control group, the dopamine agonist treated animals exhibit lower plasma triglyceride level, pro-coagulant and pro-inflammatory factor(s) level, blood pressure, and insulin resistance.

While the invention has been described in combination with embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims. All patent applications, patents, and other publications cited herein are incorporated by reference in their entireties.

1. A method of simultaneously treating hypertension, hypertriglyceridemia, a pro-inflammatory state, and insulin resistance associated with Metabolic Syndrome, said method comprising the step of administering to a patient suffering with Metabolic Syndrome a therapeutically effective amount of a central acting dopamine agonist to simultaneously treat hypertension, hypertriglyceridemia, a pro-inflammatory state, and insulin resistance.

2. The method of claim 1, wherein said method further comprises treating obesity.

3. The method of claim 1, wherein the central acting dopamine agonist is selected from the group consisting of bromocriptine, quinpirole, quinerolane, talipexole, ropinirole, apomorphine, lisuride, terguride, fenoldopam, and combinations thereof.

4. The method of claim 1, wherein the central acting dopamine agonist is administered in combination with an acceptable pharmaceutical carrier.

5. The method of claim 4, wherein the pharmaceutically acceptable carrier is selected from the group consisting of water, saline solution, gelatin, lactose, starch, magnesium stearate, talc, plant oils, gums, alcohol, petroleum jelly, and combinations thereof.

6. The method of claim 1, wherein said therapeutically effective amount of a central acting dopamine agonist ranges from 0.001 mg per kg body weight to 2.0 mg per kg body weight.

7. A method of simultaneously treating hypertension, hypertriglyceridemia, a pro-inflammatory state, and insulin resistance associated with Metabolic Syndrome, said method comprising the step of administering to a patient suffering with Metabolic Syndrome a therapeutically effective amount of a pharmaceutical composition comprising bromcriptine and a pharmaceutically acceptable carrier to simultaneously treat hypertension, hypertriglyceridemia, a pro-inflammatory state, and insulin resistance.

8. The method of claim 7, wherein said method further comprises treating obesity.

9. The method of claim 7, wherein the pharmaceutically acceptable carrier is selected from the group consisting of water, saline solution, gelatin, lactose, starch, magnesium stearate, talc, plant oils, gums, alcohol, petroleum jelly, and combinations thereof.

10. The method of claim 7, wherein said therapeutically effective amount of said pharmaceutical composition ranges from 0.001 mg per kg body weight to 0.2 mg per kg body weight.

11. The method of claim 1, wherein in said pharmaceutical composition, said bromocriptine ranges from 0.001 mg per kg body weight to 2.0 mg per kg body weight.

12. A method for simultaneously treating hypertension, hypertriglyceridemia, a pro-inflammatory state, a pro-coagulative state, and insulin resistance associated with the Metabolic Syndrome, said method comprising the step of administering to a patient suffering from Metabolic Syndrome a therapeutically effective amount of a central acting dopamine agonist to simultaneously treat hypertension, hypertriglyceridemia, a pro-inflammatory state, a pro-coagulative state, and insulin resistance.

13. The method of claim 12, wherein said central acting dopamine agonist is selected from the group consisting of bromocriptine, quinpirole, quinerolane, talipexole, ropinirole, apomorphine, lisuride, terguride, fenoldopam, and combinations thereof.

14. The method of claim 12, wherein the central acting dopamine agonist is administered in combination with an acceptable pharmaceutical carrier.

15. The method of claim 14, wherein the pharmaceutically acceptable carrier is selected from the group consisting of water, saline solution, gelatin, lactose, starch, magnesium stearate, talc, plant oils, gums, alcohol, petroleum jelly, and combinations thereof.

16. The method of claim 12, wherein said therapeutically effective amount of a central acting dopamine agonist ranges from 0.001 mg per kg body weight to 2.0 mg per kg body weight.

17. A method for simultaneously treating hypertension, a pro-inflammatory state, a pro-coagulative state, and a pro-oxidant state associated with the Metabolic Syndrome, said method comprising the step of: administering to a patient suffering from Metabolic Syndrome a therapeutically effective amount of a central acting dopamine agonist to simultaneously treat hypertension, a pro-inflammatory state, a pro-coagulative state, a pro-oxidant state, and any combination thereof.

18. The method of claim 17, wherein said central acting dopamine agonist is selected from the group consisting of bromocriptine, quinpirole, quinerolane, talipexole, ropinirole, apomorphine, lisuride, terguride, fenoldopam, and combinations thereof.

19. The method of claim 17, wherein the central acting dopamine agonist is administered in combination with an acceptable pharmaceutical carrier.

20. The method of claim 19, wherein the pharmaceutically acceptable carrier is selected from the group consisting of water, saline solution, gelatin, lactose, starch, magnesium stearate, talc, plant oils, gums, alcohol, petroleum jelly, and combinations thereof.

21. The method of claim 17, wherein said therapeutically effective amount of a central acting dopamine agonist ranges from 0.001 mg per kg body weight to 2.0 mg per kg body weight.

22. A method for simultaneously treating hypertension, a pro-inflammatory state, and a pro-coagulative state said method comprising the step of: administering to a patient suffering from hypertension, a pro-inflammatory state, and a pro-coagulative state, a therapeutically effective amount of a central acting dopamine agonist to simultaneously treat hypertension, a pro-inflammatory state, a pro-coagulative state, a pro-oxidant state, and combinations thereof.

23. The method of claim 22, wherein said central acting dopamine agonist is selected from the group consisting of bromocriptine, quinpirole, quinero lane, talipexole, ropinirole, apomorphine, lisuride, terguride, fenoldopam, and combinations thereof.

24. The method of claim 22, wherein the central acting dopamine agonist is administered in combination with an acceptable pharmaceutical carrier.

25. The method of claim 24, wherein the pharmaceutically acceptable carrier is selected from the group consisting of water, saline solution, gelatin, lactose, starch, magnesium stearate, talc, plant oils, gums, alcohol, petroleum jelly, and combinations thereof.

26. The method of claim 22, wherein said therapeutically effective amount of a central acting dopamine agonist ranges from 0.001 mg per kg body weight to 2.0 mg per kg body weight.

27. A method for treating at least one of hypertension, a pro-inflammatory state, and a pro-coagulative state, or a pro-oxidant state associated with the Metabolic Syndrome, said method comprising the step of administering to a patient suffering from Metabolic Syndrome a therapeutically effective amount of a central acting dopamine agonist to treat at least one of hypertension, a pro-inflammatory state, a pro-coagulative state, and a pro-oxidant state.

28. The method of claim 27, wherein said central acting dopamine agonist is selected from the group consisting of bromocriptine, quinpirole, quinerolane, talipexole, ropinirole, apomorphine, lisuride, terguride, fenoldopam, and combinations thereof.

29. The method of claim 27, wherein the central acting dopamine agonist is administered in combination with an acceptable pharmaceutical carrier.

30. The method of claim 29, wherein the pharmaceutically acceptable carrier is selected from the group consisting of water, saline solution, gelatin, lactose, starch, magnesium stearate, talc, plant oils, gums, alcohol, petroleum jelly, and combinations thereof.

31. The method of claim 27, wherein said therapeutically effective amount of a central acting dopamine agonist ranges from 0.001 mg per kg body weight to 2.0 mg per kg body weight.

32. A method for treating at least one of hypertension, a pro-inflammatory state, and a pro-coagulative state said method comprising the step of administering to a patient suffering from at least one of hypertension, a pro-inflammatory state, and a pro-coagulative state, a therapeutically effective amount of a central acting dopamine agonist to treat at least one of hypertension, a pro-inflammatory state, and a pro-coagulative state.

33. The method of claim 32, wherein said central acting dopamine agonist is selected from the group consisting of bromocriptine, quinpirole, quinerolane, talipexole, ropinirole, apomorphine, lisuride, terguride, fenoldopam, and combinations thereof.

34. The method of claim 32, wherein the central acting dopamine agonist is administered in combination with an acceptable pharmaceutical carrier.

35. The method of claim 34, wherein the pharmaceutically acceptable carrier is selected from the group consisting of water, saline solution, gelatin, lactose, starch, magnesium stearate, talc, plant oils, gums, alcohol, petroleum jelly, and combinations thereof.

36. The method of claim 32, wherein said therapeutically effective amount of a central acting dopamine agonist ranges from 0.001 mg per kg body weight to 2.0 mg per kg body weight.

37. A method for treating endothelial dysfunction associated with the Metabolic Syndrome, said method comprising the step of administering to a patient suffering from Metabolic Syndrome a therapeutically effective amount of a central acting dopamine agonist to treat endothelial dysfunction.

38. The method of claim 37, wherein said central acting dopamine agonist is selected from the group consisting of bromocriptine, quinpirole, quinerolane, talipexole, ropinirole, apomorphine, lisuride, terguride, fenoldopam, and combinations thereof.

39. The method of claim 37, wherein the central acting dopamine agonist is administered in combination with an acceptable pharmaceutical carrier.

40. The method of claim 39, wherein the pharmaceutically acceptable carrier is selected from the group consisting of water, saline solution, gelatin, lactose, starch, magnesium stearate, talc, plant oils, gums, alcohol, petroleum jelly, and combinations thereof.

41. The method of claim 37, wherein said therapeutically effective amount of a central acting dopamine agonist ranges from 0.001 mg per kg body weight to 2.0 mg per kg body weight.

42. A method for treating endothelial dysfunction associated with cardiovascular disease, said method comprising the step of administering to a patient suffering from endothelial dysfunction, a therapeutically effective amount of a central acting dopamine agonist to treat endothelial dysfunction.

43. The method of claim 42, wherein said central acting dopamine agonist is selected from the group consisting of bromocriptine, quinpirole, quinero lane, talipexole, ropinirole, apomorphine, lisuride, terguride, fenoldopam, and combinations thereof.

44. The method of claim 42, wherein the central acting dopamine agonist is administered in combination with an acceptable pharmaceutical carrier.

45. The method of claim 44, wherein the pharmaceutically acceptable carrier is selected from the group consisting of water, saline solution, gelatin, lactose, starch, magnesium stearate, talc, plant oils, gums, alcohol, petroleum jelly, and combinations thereof.

46. The method of claim 42, wherein said therapeutically effective amount of a central acting dopamine agonist ranges from 0.001 mg per kg body weight to 0.2 mg per kg body weight.
